(19) United States
(12) Patent Application Publication
(10) Pub. No.: US 2005/0171063 A1
(43) Pub. Date: Aug. 4, 2005

(54) Use of phosphono derivatives as anti-malarials

(76) Inventors: Pawan Malhotra, New Delhi (IN); Palakodeti Venkata Naga Dasaradhi, New Delhi (IN); Asif Mohammed, New Delhi (IN); Manzar Jamal Hossain, New Delhi (IN); Sunil Mukherji, New Delhi (IN); Venkatasamy Manivel, New Delhi (IN); Kanury Venkata Subba Rao, New Delhi (IN); Parameswaran Perunninakulath Subrayan, Dona Paula (IN); Anil Chatterji, Dona Paula (IN)

Correspondence Address: FISH & RICHARDSON PC, 225 FRANKLIN ST, BOSTON, MA, 02110, US

(21) Appl. No.: 10/893,474

(22) Filed: Jul. 16, 2004

Related U.S. Application Data
(60) Provisional application No. 60/512,906, filed on Oct. 20, 2003.

(51) Int. Cl. A61K 31/66; A61K 31/555; A61K 31/506; A61K 31/4745; A61K 31/335; A61K 31/315

(52) U.S. Cl. 514/102; 514/184; 514/305; 514/452; 514/275; 514/494

(57) ABSTRACT

The invention provides the use of phosphono derivatives of selected aliphatic acids represented by the structural formulae R—COOH, R being PO₃H₂ or CR₁R₂—PO₃H₂ where R₁/R₂ are H, OH, COOH or alkyl groups for the treatment of malaria. The use of the compound comprises administering an effective amount of said compounds for in vitro and in vivo studies using malarial parasites (Plasmodium falciparum and Plasmodium berghei).

### FIELD OF INVENTION

This invention relates to the novel phosphono derivatives as lead molecules against malaria. More specifically the present invention relates to phosphono derivatives of selected aliphatic mono- and di-carboxylic acids represented by the general formulae R—COOH, R being PO₃H₂ or CR₁R₂—PO₃ H₂ where R₁/R₂ are H, OH, COOH or alkyl groups possessing pronounced anti-malarial activity and devoid of any side effects on RBCs. More particularly this invention relates to the novel use of phosphono formic acid and phosphono acetic acid for the treatment of malaria and a process of preparing the said derivatives.

### BACKGROUND

Malaria is an important tropical parasitic disease. Relatively it kills more people than any other communicable diseases except tuberculosis. In developing countries, especially, in Africa, malaria leads to enormous loss of human lives and serious economic and medical costs. The causative agents in humans are four species of single-celled parasites, borne by mosquitoes. Among these, the parasite, P. falciparum accounts for majority of the lethal infections. About 300-400 million people come in cantact with the malarial parasite every year and about 1.7 million death have been reported every year due to malaria.

Malaria represents the toughest challenge facing modern medicine as parasite has a complex life cycle involving two hosts, human and mosquito and there is no malaria vaccine yet in sight. With the development and spread of resistance to most of current anti malarials there is a need to develop new anti malarials. PAA in plants have been shown to specifically inhibit Ct DNA polymerase (Gaikwad et al, 2002). Malaria parasite Is an apicomlexan parasite i.e. it contains a plastid like structure, which has heen shown to be crucial for the parasite survival.

Recently this plastid like organell has been shown to be an important drug target (Malhotra et al. 1997). Based on the plant study, we wanted to use PAA to inhibit plastid specific DNA polymerase. Moreover PAA/PFA has been shown to inhibit viral replication and are in use as anti-viral agent (Oberg, 1983).

Current Status of Anti-Malarial Drugs

Chloroquine (a rapid schizonticide against all infections of Plasmodium malariae and P. ovale as well as immature gametocytes of P. falciparum and not active against intrahepatic forms), Amodiaquine (an antipyretic and anti-inflammatory drug and effective than chloroquine in cleaning the parasites from the blood to enhance clinical recovery faster), Sulfadoxine—Pyrimethamine (highly active blood schizonticides against P. falciparum and less effective against other Plasmodium sp) Proguanil (a synthetic biguanide derivative of pyrimidine with marked effect on the primary tissue stages of P. falciparum, P. vivax and P. ovale), Mefloquine (a potent long acting blood schizonticide against P. falciparum and highly active against P. vivax and P. malariae. It is not gamatocytocidal and not active against the hepatic stages of malarial parasites), Quinine (a reasonable option against P. falciparum resistant to chloroquine and Sulfadrug—pyrimethamine combinations. It is extensively used in South East Asia), Halofantrine (a schizonticide that is active against all malarial parasites especially to chloroquine and sulphadrug resistant P. falciparum. It has no place in malarial control because of its high cost, variable bioavailability and cardio toxicity), Artemisinin (isolated from Artemisia annua in China and effective against P. vivax and chloroquine and Sulphadrug—pyrimethamine resistant P. falciparum. Artemisinin and its derivatives Artemether and Artesunata are the most rapidly effective antimalarial drugs).

Phosphono acetic acids in plants have been shown to specifically inhibit Ct DNA polymerase (Gaikwad et al, 2002). Malaria parasite is an apicomlexan parasite i.e. it contains a plastid like structure, which has been shown to be crucial for the parasite survival. Recently this plastid like organell has been shown to be an important drug target (Malhotra et al., 1997).

Cost Effective Factor

The direct and indirect costs of malaria in sub-Saharan Africa exceed $2 billion, according to 1997 estimates. According to UNICEF, the average cost for each nation in Africa to implement malaria control programmes is estimated to be at least $300,000 a year. This amounts to about six US cents ($0.06) per person for a country of 5 million people.

Characteristics of the New Lead Molecule

The crude extract was prepared by the enzyme-acid hydrolyzing process extracted from Mussel species belonging to family Mytilidae, found in the Ocean water of Goa, India. The mussels belonging to this family comprise of brown mussel, green mussel and other related mussels. The extract from the mussle belonging to this family showed initially a potent anti-malarial activity, at least when examined for in vitro cultures of Plasmodium falciparum in human erythrocytes. This led to an effort towards isolating and characterizing the molecular entity(ies) responsible for anti-malarial activity. An activity-guided fractionation strategy was followed in present research where a variety of chromatographic steps were employed. These included HPLC using a range of columns (hydrophobic, selective absorption, ion-exchange etc.), preparative thin layer chromatography, selective derivatization and gel filtration chromatography. Selective enrichment of activity was monitored at every step using P. falciparum culture for in vitro studies. This effort has resulted in eventually identifying an active compound that independently showed anti-malarial activity. This was followed by structure elucidation of the chemical structure of the compound. The elucidated structure was also Independently validated and found potent. Based on the plant study, we wanted to use phosphono actetic acid (PAA) to inhibit plastid specific DNA polymerase. Moreover PAA/PFA has been shown to inhibit viral replication and are in use as anti-viral agent (Oberg, 1983).

The present invention provides a extract prepared by the enzyme-acid hydrolyzing process, from a marine organism, showed initially a potent anti-malarial activity, at least when examined for in vitro cultures of Plasmodium falciparum In human erythrocytes. This led to an effort towards isolating and characterizing the molecular entity(ies) responsible for anti-malarial activity. An activity-guided fractionation strategy was followed in present research where a variety of chromatographic steps were employed. These included HPLC using a range of columns (hydrophobic, selective absorption, ion-exchange etc.), preparative thin layer chromatography, selective derivatization and gel filtration chromatography. Selective enrichment of activity was monitored at every step using P. falciparum culture for in vitro studies. This effort has resulted in eventually identifying an active compound that independently showed anti-malarial activity. This was followed by structure elucidation of the chemical structure of the compound. The elucidated structure was also independently validated and found potent. Based on the plant study, we wanted to use phosphono actetic acid (PAA) to Inhibit plastid specific DNA polymerase. Moreover PAA/PFA has been shown to inhibit viral replication and are in use as anti-viral agent (Oberg. 1983).

### OBJECTIVES OF THE INVENTION

The main object of the present invention provides phosphono derivatives of selected aliphatic acids/dicarboxylic acids having structural formula R—COOH, wherein R is PO₃H₂ or CR₁R₂—PO₃H₂, wherein R₁/R₂ are H, OH, COOH or alkyl groups against malaria.

Yet another object of the present Invention provides an alternative antimalarial drug of phosphono derivatives of selected aliphatic acids/dicarboxylic acids, especially for Plasmodium species selected from group of P. vivax, P. ovale, P. malariae, P. falciparum, P. bergei and other known plasmodia.

Still another object of the present invention provides the effect of an alternative antimalarial drug, for drug resistant plasmodium parasites selected from group comprising of P. vivax, P. ovale, P. malariae, P. falciparum, P. bergei and other known plasmodia.

Another object of the present invention provides a method of treatment or prevention of malaria by administering Phosphono derivatives selected from group comprising of aliphatic mon- and di-carboxylic acids having structural formula R—COOH, wherein R is PO₃H₂ or CR₁R₂—PO₃H₂, wherein R₁/R₂ are H, OH, COOH or alkyl groups to mammals, preferably humans.

Still another object of the present invention relates to a pharmaceutical composition for prevention or treatment of malaria by administering effective of Phosphono derivatives selected from group comprising of aliphatic mon- and di-carboxylic acids having structural formula R—COOH, wherein R is PO₃H₂ or CR₁R₂—PO₃H₂, wherein R₁/R₂ are H, OH, COOH or alkyl groups.

Yet another object of the present invention relates to use PAA and PFA and derivatives thereof to target and inhibit specific DNA polymerase of plastid like structure of parasite plasmodium in various species of plasmodium.

### SUMMARY OF THE INVENTION

The present invention relates to the anti-malarial activity of phosphono derivatives of aliphatic acids of general formulae R—COOH, R being PO3H2 or CR1R2-PO3H2 where R₁/R₂ are H, OH, COOH or alkyl groups. In particular, this relates to the anti-malarial activity of phosphono formic acid and phosphono acetic acid which show Inhibition of growth of the human malarial parasite Plasmodium falciparum in RBC cultures In vitro and mouse malarial parasite P. berghei in vivo. These compounds are lethal to the parasite, but have no effect on the RBCs and mice.

### BRIEF DESCRIPTION OF ACCOMPANYING DRAWINGS/FIGURES

FIG. 1: Growth inhibition of malaria parasite (P. falciparum) with different concentrations of phosphono acetic acid.

FIG. 2: Growth inhibition of malaria parasite (P. falciparum) with different concentrations of phosphono formic acid.

FIG. 3: Growth inhibition of mouse malaria parasite (P. berghei) with different doses of phosphono acetic acid.

FIG. 4: The effect of phosphono acetic acid on the 3D7 strain of P. falciparum

FIG. 5 Effect of PAA on survival of infected mice

### DETAILED DESCRIPTION OF THE INVENTION

Characteristics of the New Lead Molecule

The crude extract prepared by the enzyme-acid hydrolyzing process, from a marine organism, showed initially a potent anti-malarial activity, at least when examined for in vitro cultures of Plasmodium falciparum in human erythrocytes. This led to an effort towards isolating and characterizing the molecular entity(ies) responsible for anti-malarial activity. An activity-guided fractionation strategy was followed in present research where a variety of chromatographic steps were employed. These included HPLC using a range of columns (hydrophobic, selective absorption, ion-exchange etc.), preparative thin layer chromatography, selective derivatization and gel filtration chromatography. Selective enrichment of activity was monitored at every step using P. falciparum culture for in vitro studies. This effort has resulted in eventually identifying an active compound that independently showed anti-malarial activity. This was followed by structure elucidation of the chemical structure of the compound. The elucidated structure was also independently validated and found potent. Based on the plant study, we wanted to use phosphono actetic acid (PAA) to inhibit plastid specific DNA polymerase. Moreover PAA/PFA has been shown to inhibit viral replication and are in use as anti-viral agent (Oberg, 1983).

The present invention concerns uses of different phosphono derivatives of of the general formulae R—COOH, R being PO3H2 or CR1R2-PO3H2 where R₁/R₂ are H, OH, COOH or alkyl groups for the inhibition of growth of the human malarial parasite Plasmodium falciparum in RBC cultures in vitro and mouse malarial parasite P. berghei in vivo. These compounds are lethal to the parasite, but have no effect on the RBCs and mice.

In particular, the invention relates to the use of compounds; phosphono formic acid and phosphono acetic acid for the treatment of malaria.

Accordingly the main embodiment of the present invention relates to a method of treating and/or preventing malaria said method comprising of administering effective dosage of Phosphono derivatives selected from group comprising of aliphatic mon- and di-carboxylic acids having structural formula R—COOH, wherein R is PO₃H₂ or CR₁R₂—PO₃H₂, wherein R₁/R₂ are H, OH, COOH or alkyl groups to mammals, preferably humans, optionally along with acceptable additives, carriers, diluents, solvents, filters, lubricants, excipients, binder or stabilizers.

Another embodiment of the present invention relates to a pharmaceutical composition for prevention or treatment of malaria in mammals, preferably humans said composition comprising of administering effective of Phosphono derivatives selected from group comprising of aliphatic mon- and di-carboxylic acids having structural formula R—COOH, wherein R is PO₃H₂ or CR₁R₂—PO₃H₂, wherein R₁/R₂ are H, OH, COOH or alkyl groups, optionally along with acceptable additives, carriers, diluents, solvents, filters, lubricants, excipients, binder or stabilizers.

Another embodiment of the present invention relates to phosphono derivatives of the main embodiment wherein said derivatives can be administered along with zinc complexes of selected amino acids from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc (As filed in U.S. Provisional Patent Application No. 60/512,778, filed on Oct. 10, 2003).

Still another embodiment of the present invention relates to the derivatives wherein said derivatives are lethal to the malarial plasmodia selected from group comprising of P. vivax, P. ovale. P. malariae, P. falciparum, P. bergei and other known plasmodia.

Another embodiment of the present invention relates to Phosphono derivatives wherein phosphono derivatives are isolated from extract of Mussel species belonging to family Mytilidae, found in the Ocean waters of Goa, India.

Still another embodiment of the present invention relates to mussels species belonging to family Mytilidae wherein mussel species belonging to family Mytilidae are selected from group consisting of brown mussel, green mussel and other related mussels.

Yet another embodiment of the present invention relates to phosphonos derivatives wherein, the said derivatives are selected from groups comprising of aliphatic phosphono derivatives and compounds thereof.

One more embodiment of the present invention relates phosphono derivatives wherein the aliphatic acids in the said derivatives are selected from group comprising of formic acid and acetic acid and compounds thereof.

Another embodiment of the present invention relates to phosphono derivatives wherein the phosphonic acid group in the said derivatives is attached at C-1 or C-2.

Another embodiment of the present invention relates to phosphono derivatives wherein said derivatives is administered in the form of injectables, tablets, capsules, syrup, for the treatment of malaria.

Yet another embodiment of the present invention relates to additives, carriers, diluents, solvents, filters, lubricants, excipients, binder or stabilizers wherein additives, carriers, diluents, solvents, filters, lubricants, excipients, binder or stabilizers maybe selected from group consisting of lactose, mannitol, sorbitol, microcrystalline cellulose, sucrose, sodium citrate, dicalcium phosphate, magnesium stearate, calcium stearate or steorotes, talc, solid polyethylene glycols, sodium lauryl sulphate, cetyl alcohol, glyceryl monostearate or any other acceptable additives, carriers, diluents, solvents, filters, lubricants, excipients, binder or stabilizers of the similar nature alone or in a suitable combination thereof.

Still another embodiment of the present invention relates to phosophono derivatives wherein said derivatives are lethal to the parasite, but with no effect on the RBCs.

One more embodiment of the present invention relates to phosphono derivatives wherein said derivatives inhibit the growth of the malarial parasite (Plasmodium faciparum) in RBC cultures.

Yet another embodiment of the present invention relates to phosphono derivatives wherein derivative phosphono acetic acid kills the parasites by disintegrating trophozoites.

Another embodiment of the present invention relates to phosphono acetic acid wherein phosphono acetic acid in the range of about 1 to 5 μM of inhibits growth of P. facliparum by about 100%.

Yet another embodiment of the present invention relates to phosphono formic acid wherein phosphono formic acid in the range of 5 to 10 μM inhibits growth of P. falciparum by about 90%.

One more embodiment of the present invention relates to phosophono formic acid wherein phosphono formic acid in the range of 5 to 10 μM inhibits growth of P. falciparum by about 70%.

Another embodiment of the present invention relates phosphono formic acid wherein about 1 to 50 mg/Kg of phosphono formic acid inhibits growth of P. berghei by about 90%.

Still another embodiment of the present invention relates phosphono formic acid wherein about 1 to 50 mg/kg of phosphono formic acid inhibits growth of P. berghei by about 60%.

Still another embodiment of the present invention relates phosphono acetic acid acid wherein phosphono acetic acid at a dose in the range of about 1 to 50 mg/kg enhances the survival by about 60%.

Still another embodiment of the present invention relates phosphono derivatives wherein said derivatives can be administered along with other antimalarial drugs.

One more embodiment of the present invention relates to antimalarial drugs, wherein the anti malarial drugs may be selected from group consisting of Chloroquine and its derivatives. Amodiaquine, Sulfadoxine, Pyrimethamine and its derivatives, Proguanil, Mefloquine, Quinine, Halofantrine, Artemisinin, Artemether and Artesunata and their derivatives or other known antimalarial drugs.

The following examples are given by way of illustration of the present invention and therefore should not be construed to limit the scope of the present invention.

### EXAMPLES

### Example 1

Extraction of Phosphono derivatives from Mussel Extract

Mussel hydrolysate was lyophilized to get crude solid from which methanolic extract was obtained by adding 150 ml methanol and stirring for 90 mins at RT. Filtered with filter paper. The filtrate was labeled as AcM. The AcM fraction was subjected to HPLC on a RP-C18 column using a linear gradient of 0-60% B acetonitrile over forty minutes. The peak eluting at void volume (10 mins) was collected and lyophilized. The crude solid was dissolved in 60 ml milliQ water and was fractionated on sephadex-G15 column and eluted with H2O. Fraction 6-11 were pooled and lyophilized and labeled as P2N. P2N was further purified using prep-TLC on silica gel with BAW=4:1.5:1 as the mobile phase. Two fractions labled K-1-1 and K-1-2 were obtained alter extracting silica gel with 0.01N HCl. Lyophilized to get solid and activity was found in K-1-2. K-1-2 was further sub-fractionated on HSF5 RP column using water as the mobile phase under isocratic condition to get K1-1-2/1 and K-1-2/2 and both were given anti-malaria activity. The fraction K-1-2/1 were found to be Amino Acid-zinc complexes, whereas K-1-2/2 were found to be phosphono derivatives. Both these fractions i.e K-1-2/1 and K-1-2/2 showed anti-malarial activity. Further K-1-2/2 was determined by mass spectrometry confirmed it to be phosphono derivatives. Finally the activity for the Isolated compound from the mussle extract was reproduced by commercially available synthetic phosphono derivatives like phosphono acetic acid and phosphono formic acid (These are commercially available from Sigma-Aldrich, Catalogue No. 440% 78-0 and 28427-0). The Mass spectrometry between the isolated phosphono derivatives from the mussel extract and the commercially available phosphono derivatives matched perfectly to prove the findings from the mussel extract. In other words the mussel extract fraction K-1-2/2 were similar to commercial phosphono compounds in their mass spectrometry analysis.

### Example 2

Phosphono acetic acid was dissolved in normal saline and filter sterilized. The compound was added to the parasite culture at different concentrations ranging from 1-400 μm. The compound phosphono acetic acid was tested at the indicated doses using the experimental protocol as described below:

Protocol for Testing the Effect of Drug on P. falciparum for In Vitro Studies

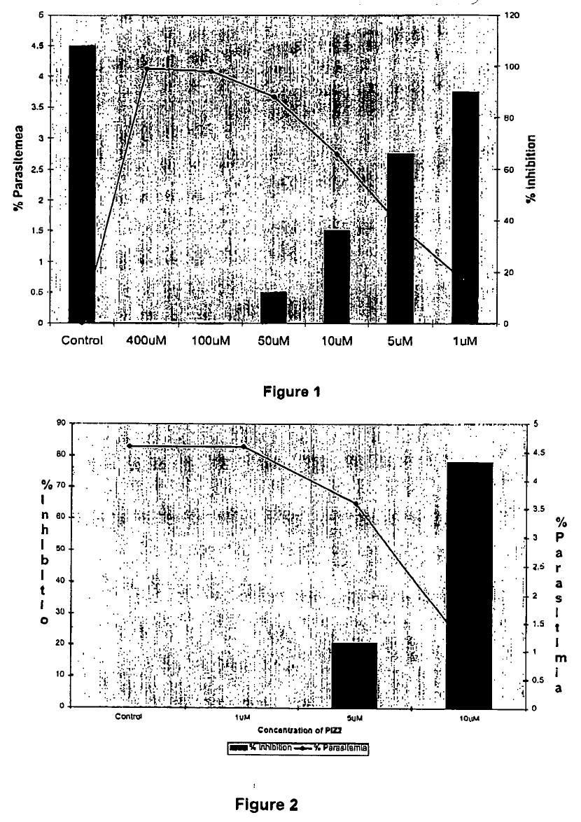

The P. falciparum cultures were synchronized at first by sorbitol treatment. The compound of various concentrations was added to the 200 μl of synchronized P. falciparum culture (1% parasitemia). The parasitemia was checked by making Giemsa stained smear after 48 hrs of incubation at 37° C. The growth of P. falciparum was inhibited in dose-dependent manner, where 1 μM concentration yielded >90% inhibition (Plate 1). The resulting dose-dependent response obtained is shown in FIG. 1. The bars represent the percent inhibition, whereas, the blue curve indicates the percentage of parasitemia. From the graph, the concentration required for half-maximal (LD₅₀) inhibition is calculated to be about 7.5 μM (FIG. 1).

### Example 3

Phosphono formic acid was dissolved in normal saline and filter sterilized. The compound was added to the parasite culture at different concentrations ranging from 1-10 μM. The growth of P. falciparum was inhibited in dose-dependent manner, where 10 μM concentration yielded −70% inhibition (FIG. 2).

### Example 4

Effect of Phosphono Acetic Acid on P. berghei Infected Mice (In Vivo Model)

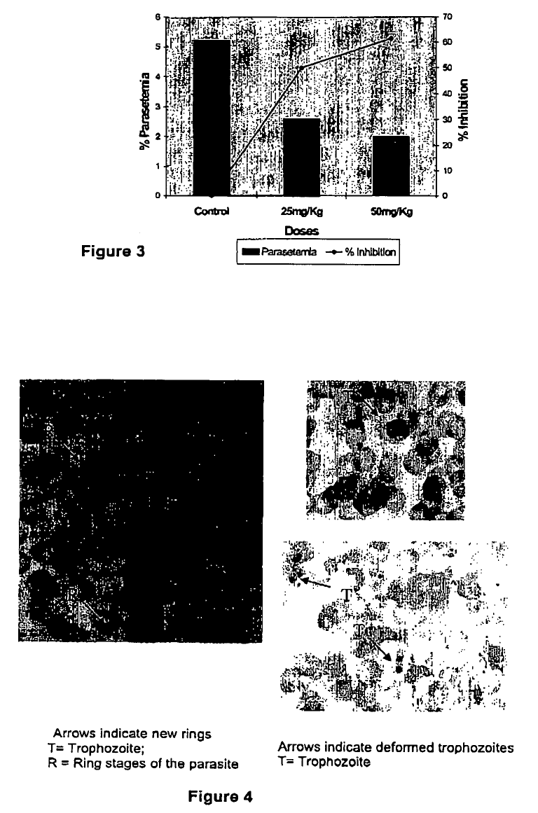

BALB/c mice (4-6 weeks old) were infected with 10⁵ P. berghei parasites/mouse. After four days of infection, when phosphono acetic acid (dissolved in saline and filter sterilized) at a dose of either 25 mg/kg body weight, or 50 mg/kg body weight. The injection of phosphono acetic acid was continued, once a day, for four days. After the fourth day of injection, parasitemea was determined by making Geimsa stained blood smears from the mice. Survival rate of control and infected mice was also calculated The growth of P. berghei was Inhibited in dose-dependent manner, where 50 mg/kg of body weight showed −60% inhibition (FIG. 3).

### Example 5

The effect of Phosphono Acetic Acid on the 3D7 Strain of P. falciparum

Giemsa-stained slides prepared for the experiment were examined under a microscope and represented in Plate 4. In this experiment, disintegrated trophozoltes are seen upon the addition of PAA. It is clear that PAA actually kills the parasite rather than simply inhibiting their growth (FIG. 4).

### Example 6

Effect of PAA on Survival of Infected Mice

The P. falciparum cultures were synchronized at first by sorbitol treatment.

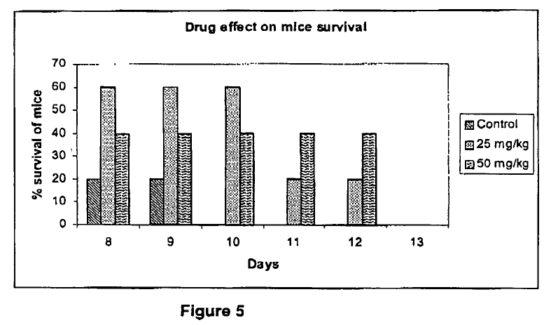

The compound of various concentrations was added to the 200 μl of synchronized P. falciparum culture (1% parasitemia). The parasitemia was checked by making Giemsa stained smear after 48 hrs of incubation at 37° C. The compound, PAA was used at the doses of 25 mg/kg and 50 mg/kg body weight. The control group indicates the group of mice infected with the parasite, but with no drug given. Each group contained ten mice, and the percent of mice surviving at the Indicated days is shown in FIG. 5.

### ADVANTAGES OF THE PRESENT INVENTION

1) The present active compound for malaria is relatively cheap and can readily be prepared in bulk.

2) It adds to the existing list of anti-malaria compounds, and may be used in conjunction with the other conventional drugs such as chlroquine, mefloquine etc.

3) It may also be useful against drug resistant malarial parasite.

### REFERENCES

1. Bradley, D. J. Warhurst, D. C., Br. Med. J., 310, 709-714, 1995.
2. Datta, A. K. and Hood, R. E., Virology, 114, 52-59, 1981.
3. Dorn, A., Stoffel, R., Matile, H., Bubendorf, A., Ridley, R. G., Nature, 374, 269-271, 1995.
4. Malhotra, P, Dasaradhi, P. V. N. Kumar, Amit, Mohammed Asif, Agarwal, N., Bhatnagar, R. K. and Chauhan, V. S., Mol. Microbiol. 45, 1245-1254, 2002
5. McConkey, G. A., Rogers, M. J. and McCutchan, T. F., J. Biol. Chem., 272, 2046-2049, 1997.
6. Murphy, G., Basri, H., Pumomo, Lancet, 341, 96-100, 1993.
7. Oberg, Bo, Pharmac. Ther., 19, 387-415, 1983.
8. Slater, A. F. G., Pharmac. Ther., 57, 203235, 1993.
9. Tiffert, T., Ginsburg, H., Krugliak, M., Elford, B. C. and Lew, V. L., Proc. Natl. Acad Sci. USA, 97, 331-336, 2000.
10. Wernsdorfer, W. H., Payne, D., Pharmac. Ther., 50, 95-121, 1991.
11. White, N. J., Br. J. Clin. Pharmacol., 34, 1-10, 1992.

1. A method of treating and/or preventing malaria said method comprising of administering effective dose of Phosphono derivatives selected from group consisting of aliphatic mon- and di carboxylic acids having structural formula R—COOH, wherein R is PO₃H₂ or CR₁R₂—PO₃H₂, wherein R₁/R₂ are H, OH, COOH or alkyl groups to mammals, preferably humans, optionally along with acceptable additives, carriers, diluents, solvents, filters, lubricants, excipients, binder or stabilizers.

2. A method as claimed in claim 1, wherein said derivatives are lethal to the malarial plasmodia selected from group comprising of P. vivax, P. ovale, P. malariae, P. falciparum, P. bergei and other known plasmodia.

3. A method as claimed in claim 1 wherein said derivatives can be administered along with zinc complexes of selected amino acids from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc ((As filed in U.S. Provisional Patent Application No. 60/512,778, filed on Oct. 10, 2003)

4. A method as claimed in claim 1 wherein said derivatives can be administered along with other antimalarial drugs.

5. A method as claimed in claim 4, wherein other anti malarial drugs may be selected from group consisting of Chloroquine and its derivatives, Amodiaquine, Sulfadoxine, Pyrimethamine and its derivatives, Proguanil, Mefloquine, Quinine, Halofantrine, Artemisinin, Artemether and Artesunata and their derivatives and known antimalaria drugs.

6. A method as claimed in claim 1, wherein Phosphono derivatives are isolated from extract of Mussel species belonging to family Mytilidae, found in the Ocean water of Goa, India.

7. A method as claimed in claim 6, wherein mussel species belonging to family Mytilidae are selected from group consisting of brown mussel, green mussel and other related mussels.

8. A method as claimed in claim 1, wherein, the said derivatives are selected from groups comprising of aliphatic phosphono derivatives and compounds thereof.

9. A method as claimed in claim 1 wherein the aliphatic acids in the said derivatives are selected from group comprising of formic acid and acetic acid and compounds thereof.

10. A method as claimed in claim 1 wherein the phosphonic acid group in the said derivatives is attached at C-1 or C-2.

11. A method as claimed in claim 1 wherein said derivatives is administered in the form of injectables, tablets, capsules, syrup, for the treatment of malaria.

12. A method as claimed in claim 1, wherein additives, carriers, diluents, solvents, filters, lubricants, excipients, binder or stabilizers maybe selected from group consisting of lactose, mannitol, sorbitol, microcrystalline cellulose, sucrose, sodium citrate, dicalcium phosphate, magnesium stearate, calcium stearate or steorotes, talc, solid polyethylene glycols, sodium lauryl sulphate, cetyl alcohol, glyceryl monostearate or any other acceptable additives, carriers, diluents, solvents, filters, lubricants, excipients, binder or stabilizers of the similar nature alone or in a suitable combination thereof.

13. A method as claimed in claim 1 wherein said derivatives are lethal to the parasite, but with no effect on the RBCs.

14. A method as claimed In claim 1 wherein said derivatives inhibit the growth of the malarial parasite (Plasmodium falciparum) in RBC cultures.

15. A method as claimed in claim 1 wherein of phosphono acetic acid in the range of about 1 to 5 μM inhibits growth of P. facliparum by about 100%.

16. A method as claimed in claim 1 wherein phosphono formic acid in the range of about 5 μM to 10 μM inhibits growth of P. falciparum by about 90%.

17. A method as claimed in claim 16 wherein phosphono formic acid in the range of about 5 μM to 10 μM inhibits growth of P. falciparum by about 70%.

18. A method as claimed in claim 1 wherein about 1 mg to 50 mg/Kg of phosphono formic acid Inhibits growth of P. berghei by about 90%.

19. A method as claimed in claim 18 wherein about 1 mg to 50 mg/kg of phosphono formic acid inhibits growth of P. berghei by about 70%.

20. A method as claimed in claim 1 wherein phosphono acetic acid at a dose in the range of about 1 to 50 mg/kg enhances the survival by about 60%.

21. A pharmaceutical composition for prevention or treatment of malaria in mammals, preferably humans said composition comprising of administering effective of Phosphono derivatives selected from group comprising of aliphatic mon- and di-carboxylic acids having structural formula R—COOH, wherein R is PO₃H₂ or CR₁R₂—PO₃H₂, wherein R₁/R₂ are H, OH, COOH or alkyl groups, optionally along with acceptable additives, carriers, diluents, solvents, filters, lubricants, excipients, binder or stabilizers.

22. A composition as claimed in claim 21, wherein said derivatives are lethal to the malarial plasmodia selected from group comprising of P. vivax, P. ovale, P. malariae, P. falciparum, P. bergei and other known plasmodia.

23. A composition as claimed in claim 21 wherein said derivatives can be administered along with zinc complexes of selected amino acids from D or L isomers of proline, lysine, histidine, glycine, arginine and tryptophan or their various hydroxyl, amino, alkyl and carboxyl derivatives and zinc chloride, zinc acetate or other pharmacologically acceptable salts of zinc (As filed in U.S. Provisional Patent Application No. 60/512778, filed on Oct. 10, 2003)

24. A composition as claimed in claim 21 wherein said derivatives can be administered along with other antimalarial drugs.

25. A composition as claimed in claim 24, wherein other anti malarial drugs may be selected from group consisting of Chloroquine and its derivatives, Amodiaquine, Sulfadoxine, Pyrimethamine and its derivatives, Proguanil, Mefloquine. Quinine, Halofantrine, Artemisinin, Artemether and Artesunata and their derivatives or other known antimalarial drugs.

26. A composition as claimed in claim 21, wherein Phosphono derivatives are isolated from extract of Mussel species belonging to family Mytilidae, found in the Ocean waters of Goa, India.

27. A composition as claimed in claim 26, wherein mussels species belonging to family Mytilidae are selected from group consisting of brown mussel, green mussel and other related mussels.

28. A composition as claimed in claim 21 wherein, the said derivatives are selected from groups comprising of aliphatic phosphono derivatives and compounds thereof.

29. A composition as claimed in claim 21 wherein the aliphatic acids in the said derivatives are selected from group comprising of formic acid and acetic acid and compounds thereof.

30. A composition as claimed in claim 21 wherein the phosphonic acid group in the said derivatives is attached at C-1 or C-2.

31. A composition as claimed in claim 21 wherein said derivatives is administered in the form of injectables, tablets, capsules, syrup, for the treatment of malaria.

32. A composition as claimed in claim 21, wherein additives, carriers, diluents, solvents, filters, lubricants, excipients, binder or stabilizers maybe selected from group consisting of lactose, mannitol, sorbitol, microcrystalline cellulose, sucrose, sodium citrate, dicalcium phosphate, magnesium stearate, calcium stearate or steorotes, talc. solid polyethylene glycols, sodium lauryl sulphate, cetyl alcohol, glyceryl monostearate or any other acceptable additives, carriers, diluents, solvents, filters, lubricants, excipients, binder or stabilizers of the similar nature alone or in a suitable combination thereof.

33. A composition as claimed in claim 21 wherein said derivatives are lethal to the parasite, but with no effect on the RBCs.

34. A composition as claimed in claim 21 wherein said derivatives inhibit the growth of the malarial parasite (Plasmodium falciparum) in RBC cultures.

35. A composition as claimed in claim 21 wherein phosphono acetic acid in the range of about 1 μM to 5 μM inhibits growth of P. facliparum by about 100%.

36. A composition as claimed in claim 21 wherein phosphono formic acid in the range of 5 μM to 10 μM inhibits growth of P. falciparum by about 90%.

37. A composition as claimed in claim 36 wherein phosphono formic acid in the range of 5 μM to 10 μM inhibits growth of P. falciparum by about 70%.

38. A composition as claimed in claim 21 wherein about 1 mg to 50 mg/Kg of phosphono formic acid inhibits growth of P. berghei by about 90%.

39. A composition as claimed in claim 38 wherein about 1 mg to 50 mg/kg of phosphono formic acid inhibits growth of P. berghei by about 60%.

40. A composition as claimed in claim 21 wherein phosphono acetic acid at a dose of 50 mg/kg enhances the survival by about 60%.
